(19) United States
(12) Patent Application Publication
(10) Pub. No.: US 2023/0059281 A1
(43) Pub. Date: Feb. 23, 2023

(54) EAR CANAL MEDICATION APPLICATOR

(71) Applicant: Thomas W. PFENNIG, Ada, MI (US)

(72) Inventor: Thomas W. PFENNIG, Ada, MI (US)

(21) Appl. No.: 17/889,318

(22) Filed: Aug. 16, 2022

Related U.S. Application Data
(60) Provisional application No. 63/234,584, filed on Aug. 18, 2021.

(51) Int. Cl. A61M 31/00

(52) U.S. Cl. CPC A61M 31/00; A61M 2210/0662

(57) ABSTRACT

An ear canal medication applicator including a tympanogram ear canal occlusion earpiece operatively connected to a bulb insufflator through a body, and a syringe operatively connected to an IV line, wherein the syringe is operatively attached to the body and the IV line is threaded through a portion of the tympanogram ear canal occlusion earpiece. A method of treating the ear canal, by providing a dual application of medication at two different locations in the ear canal of an individual and treating the ear canal.

### BACKGROUND OF THE INVENTION

### 1. Technical Field

The present invention relates to compositions and methods for treating ears externally.

### 2. Background Art

The ear canal is a pathway that runs from the outer ear (pinna) to the middle ear (ear drum). The ear canal is a very unusual skin canal that grows out at about the speed of fingernails growing, and as long as the canal's cleaning system works, there are generally few problems for individuals. It picks up cerumen (ear wax) as a passenger as skin grows towards the external meatus. In the ear canal as everywhere else on the skin, there are bacteria and fungus. All of the pathogens that are commonplace for external canal concerns are related to Staph infections, Pseudomonas infections, and fungus. All of these, but in particular the fungal canal concerns, are only opportunistic. Warm conditions, moist conditions, organic material, and time equates to symptoms in the ear canal.

There are different issues that can occur with the external ear canal. Patients present with a myriad of diagnoses to explain the large variety of symptoms including aural fullness, pain or pressure, draining ears, as well as commonly itching ears. Often times these are attributed to cerumen, but it is very commonplace that the canals are shiny and clean with no debris at all within the canal. Even if the ear canal does have cerumen debris, itching of the ear canal was often times what was prompting more Q-tip use and thus the development of the impaction. The bothersome itching ear complaint is often the source of “eczematoid otitis externa” and “fungal otitis externa”.

Current treatment of the external ear includes either expensive or painful ototopical drops that are used frequently and include antibiotic with or without steroid preparations. Some of these preparations are also ototoxic and have limited uses. Some people have an immediate, short term acute need but often times more chronic needs for ear canal complaints. These include but are not limited to 1) swimmers (not necessarily from dirty water, but the presence of too much water and too much cleaning), 2) hearing aid wearers because of the inclusion of the ear canal causing increasing warmth and moisture to the air canal, 3) earplugs and earbuds, the use of the ear canal as a location for a multitude of music communication and health devices, 4) Q-tip overuse, the perceived solution now becomes the problem of excessive cleaning of the canal, and 5) people that have had ear surgery related to the tympanic membrane, elderly people where the canal begins to collapse, inherently narrow canals, and other canal abnormalities.

Currently, an insufflator powder applicator can be used with an operating otoscope for direct visualization to apply antibiotic and anti-fungal powder (such as chloramphenicol, amphotericin, and sulfa) to a person's eardrum and the first centimeter of the ear canal closest to the drum. An angiocatheter tip can also be used through an otoscope for direct visualization to apply a bead of anti-fungal steroid cream for about 5 mm along the ear canal on the roof. Both of these techniques currently require visualization to perform. Other devices and/or therapies that are widely used or are in development and use include Gentian violet applicators, multidose powder applicators, and recently, the use of a Angiocaths (also known as IV catheter), used off label, from sizes from 20 to 24 demonstrated for direct visualization only for the placement of a new ciprofloxin gel, Otiprio.

There exists a need for a delivery device that manages both the eardrum and external ear concerns. This need is for a dual application of two styles of medications because of the need to treat completely to the eardrum and to treat without the need for visualization. Currently, no available treatment program exists that applies these medications into two distinct areas of the ear canal.

### SUMMARY OF THE INVENTION

The present invention provides for an ear canal medication applicator including a tympanogram ear canal occlusion earpiece operatively connected to a bulb insufflator through a body, and a syringe operatively connected to an IV line, wherein the syringe is operatively attached to the body and the IV line is threaded through a portion of the tympanogram ear canal occlusion earpiece.

The present invention provides a method of treating the ear canal, by providing a dual application of medication at two different locations in the ear canal of an individual and treating the ear canal.

### DESCRIPTION OF THE DRAWINGS

Other advantages of the present invention are readily appreciated as the same becomes better understood by reference to the following detailed description when considered in connection with the accompanying drawings wherein:

FIG. 1 is a side view of the ear canal medication applicator; and

FIG. 2 is a front perspective view of the ear canal medication applicator.

### DETAILED DESCRIPTION OF THE INVENTION

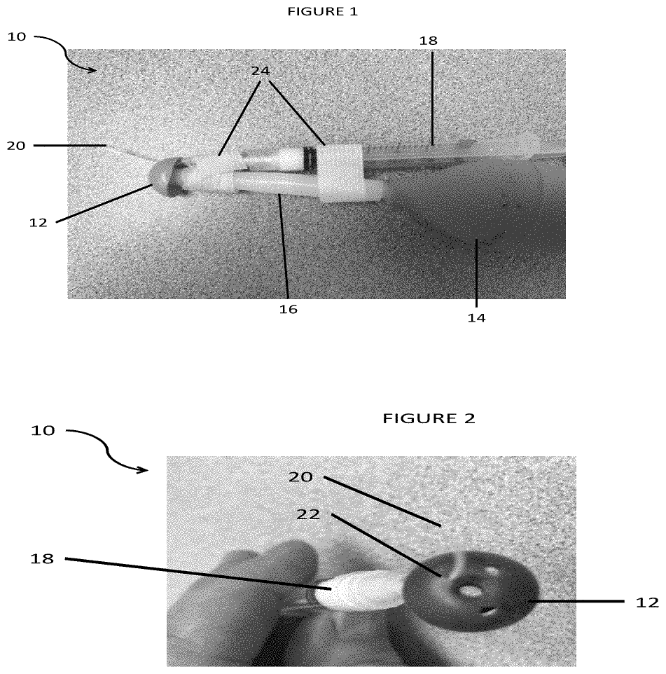

The present invention generally provides for an ear canal medication applicator, shown at 10 in FIGS. 1 and 2, for treating both the ear canal and tympanic membrane. The present invention solves the problem of the ability to apply medications in a specific location with consistency and safety without direct visualization in the ear canal.

The ear canal medication applicator 10 includes a tympanogram ear canal occlusion earpiece 12 operatively connected to a bulb insufflator 14 through a body 16, and a syringe 18 operatively connected to an IV line 20, wherein the syringe 18 is operatively attached to the body 16 and the IV line 20 is threaded through a portion of the tympanogram ear canal occlusion earpiece 12.

The tympanogram ear canal occlusion earpiece 12 is preferably made of silicone but can be made of other materials such as metal. It limits how far the ear canal medication applicator 10 extends into the ear to make sure it gets close to the ear drum and deep enough in the ear canal to achieve a treatment close to the ear drum but not too close to potentially cause damage to the ear.

The bulb insufflator 14 is connected to the tympanogram ear canal occlusion earpiece 12 through the body 16. Both the bulb insufflator 14 and the body 16 can be made of plastic or latex. The bulb insufflator is preferably used to deliver a powder with anti-fungal and antibiotic properties, such as chloramphenicol, amphotericin, and with or without sulfa, with a puff action around the ear drum. Other medications are further listed below. The bulb insufflator 14 can be preloaded with medication (such as a specific volume for single doses or multiple doses) or can be loaded by a medical professional.

The syringe 18 can be a 2-3 mL syringe, but other sizes can be used to accommodate different administrations of medications. The syringe 18 can contain medication for application medial to an obstructed area of an infected and swollen ear canal. The syringe 18 is operatively connected to the IV line 20, which can be a 22-gauge or 24-gauge angiocath to push medication through to a desired portion of the ear canal and has advantages of being narrow and non-traumatizing. The IV line 20 can be threaded through a hole 22 in the tympanogram ear canal occlusion earpiece 12 to secure it (shown in FIG. 2), and the IV line 20 extends at least some distance (such as 2-25 mm, the ear canal is generally 25 mm in length) from the tympanogram ear canal occlusion earpiece 12. Additional holes 22 can be included (2 mm diameter) to allow for airflow such that powder can be more comfortably puffed into the ear canal without discomfort related to pressure on the ear drum. The syringe 18 is also operatively connected to the body 16 by any securing mechanism 24, such as tape or plastic. The syringe can be used to deliver medication such as anti-fungal cream in the ear canal, or other medications listed below. The syringe 18 can be preloaded with medication (such as a specific volume for single doses or multiple doses) or can be loaded by a medical professional.

The ear canal medication applicator 10 can be used to apply various medications in addition to those specifically described above such as, but not limited to, neomycin drops, polymyxin drops, ciprofloxacin, dexamethasone, tobramycin, gentamicin, nystatin, acetic acid, boric acid, alcohol drops, gentian violet antifungal, multiple topical steroids, and combinations thereof. Otic powder can be used having antibiotics and antifungals such as 46.7% chloramphenicol, 46.7% sulfamethoxazole, and 5.6% amphotericin. These medications can be applied to just the outer part of the ear in an attempt to treat a problem that is starting of 2.5 cm away. Dosages can be given using appropriate volume, such as 0.2 mL.

The present invention provides a method of treating the ear canal, by providing a dual application of medication at two different locations in the ear canal of an individual and treating the ear canal. More specifically, the method includes inserting the IV line 20 in the ear canal, inserting the tympanogram ear canal occlusion earpiece 12 in the ear canal, applying medication through the IV line 20 with the syringe 18, and applying medication through the tympanogram ear canal occlusion earpiece 12 with the bulb insufflator 14. The method can be used to treat infections caused by many different sources, such as Pseudomonas aeruginosa or Staphylococcus aureus.

Preferably the ear canal medication applicator 10 is disposable and able to be used outside of a doctor's office, emergency room, or primary care office by the patient or patient's family or caregiver. The present invention allows for a more consistent and less obtrusive way to resolve acute symptoms or control chronic symptoms. For acute symptoms, patients can be treated with one or two applications to resolve ear issues, and for chronic concerns the applications are only required on a range between every three- and six-month intervals.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. An ear canal medication applicator, comprising a tympanogram ear canal occlusion earpiece operatively connected to a bulb insufflator through a body, and a syringe operatively connected to an IV line, wherein said syringe is operatively attached to said body and said IV line is threaded through a portion of said tympanogram ear canal occlusion earpiece.

2. The ear canal medication applicator of claim 1, wherein said tympanogram ear canal occlusion earpiece is made of a material chosen from the group consisting of silicone and metal.

3. The ear canal medication applicator of claim 1, wherein said bulb insufflator is made of a material chosen from plastic and latex.

4. The ear canal medication applicator of claim 1, wherein said bulb insufflator delivers an anti-fungal and antibiotic powder.

5. The ear canal medication applicator of claim 1, wherein said bulb insufflator delivers a medication chosen from the group consisting of chloramphenicol, amphotericin, neomycin drops, polymyxin drops, ciprofloxacin, dexamethasone, tobramycin, gentamicin, nystatin, acetic acid, boric acid, alcohol drops, gentian violet antifungal, multiple topical steroids, otic powder, and combinations thereof.

6. The ear canal medication applicator of claim 5, wherein said medication is preloaded in said bulb insufflator.

7. The ear canal medication applicator of claim 1, wherein said body is made of a material chosen from the group consisting of plastic and latex.

8. The ear canal medication applicator of claim 1, wherein said syringe is further defined as a 2 to 3 mL syringe.

9. The ear canal medication applicator of claim 1, wherein said syringe delivers an anti-fungal cream in an ear canal.

10. The ear canal medication applicator of claim 1, wherein said syringe delivers medication in an ear canal chosen from the group consisting of chloramphenicol, amphotericin, neomycin drops, polymyxin drops, ciprofloxacin, dexamethasone, tobramycin, gentamicin, nystatin, acetic acid, boric acid, alcohol drops, gentian violet antifungal, multiple topical steroids, otic powder, and combinations thereof.

11. The ear canal medication applicator of claim 10, wherein said medication preloaded in said syringe.

12. The ear canal medication applicator of claim 1, wherein said IV line is chosen from the group consisting of 22-gauge angiocath and 24-gauge angiocath.

13. The ear canal medication applicator of claim 1, wherein said IV line is threaded through a hole in said tympanogram ear canal occlusion earpiece and extends a distance therefrom.

14. The ear canal medication applicator of claim 1, further including at least one hole allowing for airflow.

15. The ear canal medication applicator of claim 1, wherein said syringe is operatively connected to said body by a securing mechanism chosen from the group consisting of tape and plastic.

16. The ear canal medication applicator of claim 1, wherein said ear canal medication applicator is disposable.

17. A method of treating the ear canal, including the steps of:
providing a dual application of medication at two different locations in an ear canal of an individual; and
treating the ear canal.

18. The method of claim 17, wherein said providing step is further defined as delivering a powder with anti-fungal and antibiotic properties to an area around an ear drum and delivering an anti-fungal cream in an ear canal.

19. The method of claim 17, wherein said providing step is further defined as using an ear canal medication applicator by inserting an IV line in the ear canal, inserting a tympanogram ear canal occlusion earpiece in the ear canal, applying medication through the IV line with a syringe, and applying medication through the tympanogram ear canal occlusion earpiece with a bulb insufflator.

20. The method of claim 19, wherein the medication is chosen from the group consisting of chloramphenicol, amphotericin, neomycin drops, polymyxin drops, ciprofloxacin, dexamethasone, tobramycin, gentamicin, nystatin, acetic acid, boric acid, alcohol drops, gentian violet antifungal, multiple topical steroids, otic powder, and combinations thereof.

21. The method of claim 17, wherein said treating step is further defined as treating an infection caused by a bacteria chosen from the group consisting of Pseudomonas aeruginosa and Staphylococcus aureus.
